(19) United States
(12) Patent Application Publication
(10) Pub. No.: US 2002/0187478 A1
(43) Pub. Date: Dec. 12, 2002

(54) Rapid pharmaceutical component screening devices and methods

(76) Inventor: Winthrop D. Childers, San Diego, CA (US)

Correspondence Address: HEWLETT-PACKARD COMPANY, Intellectual Property Administration, P.O. Box 272400, Fort Collins, CO, 80527-2400, US

(21) Appl. No.: 09/878,108

(22) Filed: Jun. 7, 2001

(51) Int. Cl. C12Q 1/68

(52) U.S. Cl. 435/6

(57) ABSTRACT

An automated method and device for analyzing biological samples such as substances containing cellular material in which at least one jetting device is activated to dispense a volume of a potential pharmaceutically active agent into contact with a defined volume of the biological sample. Data pertaining to changes detected in the defined volume is captured and analyzed. The potential pharmaceutically active agent or multiple agents are contained in a cartridge removably insertable in the device employed. Typically, the volume of and arrangement of potentially pharmaceutically active agent can be varied over multiple sample volumes to provide arrayed or factorially derived analysis.

### FIELD OF THE INVENTION

The present invention relates to methods for analyzing and screening biological samples containing cellular material against at least one pharmaceutical or potentially bio-active component to ascertain efficacy and or toxicity. More particularly, the present invention is directed to automated methods for analyzing and screening biological samples containing cellular material in which discrete volumes of at least one potential pharmaceutically active component is introduced into contact with an array of substances containing cellular material through a liquid jetting device such as an ink jet device. The present invention is also directed to a device for accomplishing such analysis as well as component parts for use therein.

### BACKGROUND OF THE INVENTION

The need and desire to analyze and ascertain composition and characteristics of biological material such as biologically derived molecules is well documented. Similarly there exists a need to analyze and test larger specimens containing whole cells or identifiable components of cells particularly to ascertain effectiveness of materials employed with or against them.

In molecular assays, biological or chemical reagents (commonly referred to as “probes”) are often used as reagents in detection of other target biological or chemical compounds such as viruses or bacteria within a specimen under test. In such methods any such target compounds existing in the specimen can be identified through controlled exposure of the specimen to the probes and the detection of DNA hybridization or antibody-antigen synthesis. Underpinning such procedures is the knowledge and understanding of expected reactions which are likely to occur when a specimen is exposed to a known probe. Distinct variations of the reagent probes can be specifically formulated to detect particular target compounds.

In such procedures, probes may contain biological material containing target DNA of up to a thousand base pair in length. Upon controlled exposure of the specimen with the target DNA, the specimen and the target bind together or hybridize in a detectable manner. The target DNA can be labeled or modified in a manner which will assist in detection. To perform a test using a probe, a small quantity of the probe in the solution is dispensed on to a glass slide in a process referred to as “spotting”. The specimen is exposed to the spotted probe and permitted to hybridize. Excess specimen material is removed and any hybridization is readily detected.

In order to streamline and speed the spotting process, various modifications in dispensing technique have been proposed. For instance, micropipetting techniques have been proposed in which in which the micropipette is manipulated into position and visually monitored to estimate the volume of the droplet to be administered. Such techniques permit arrayed analysis of multiple specimens. Such methods are predicted upon knowledge of predictable interactions which, when evidenced, have been demonstrated to indicate the presence of the desired or targeted marker, antigen, DNA sequence or the like. These methods and devices have not been directed to evaluation of unpredictable or unknown interactions or reactions as would occur in procedures to screen new or untried compounds, either alone or in various combinations, for pharmacological or pharmaceutical efficacy. There still exists a need for rapid reproducible, volumetrically accurate dispensing techniques which can position dispensed material in a precise dimensionally defined matrix.

In order to address this need, various delivery devices employing “ink jet” technology have been proposed for use in various solid state oligomeric synthesis reactions as would be used in the DNA probe procedures outlined. Such methods and devices are directed to a chemical synthesis in which one or more chemical reagents are delivered to various specific sites on a solid support surface using a droplet generator such as an ink jet device. The solid support surfaces typically have opposed first and second surfaces connected by a series of channels, pores or the like to support various reactant species such as those amenable to solid state peptide synthetic reactions. Typically these reactions involve synthesis of polymers of nucleotides (oligonucleotides or nucliec acids), peptides, protein, peptide nucleic acids and other polymeric species synthesizable by iterative addition of synthons to adducts on the reaction surface. In such processes, chemical libraries of compounds of precisely known chemical structure are generated and collected. Once these libraries have been generated, the libraries or library subsets may be used in subsequent conventional biochemical assay techniques.

It has also been proposed that chemical processes involving reactions of organic polymers such as oligonucleotides be performed with the oligonucleotides attached to solid support surfaces. Reactant solutions are deposited onto functionalized binding sites using a piezoelectric pump such as those employed in ink jet printing technology. A dispensing device ejects volumes of each of the four standard nucleotides and a suitable coupling reagent in order to determine nucleotide sequencing of a target nucleic acid attached to the support surface.

Oligonucleotide synthesis employing ink jet type dispensing devices have also been proposed to create an array of microdrop-sized loci of synthesized oligonucleotides. The dispensing apparatus can be employed to apply a first reagent capable of covalently bonding to the substrate to which it is applied. Displacement of the substrate site relative to the dispensing apparatus is then accomplished and at least one microdrop of either a first or a second reagent can be applied. The process can be repeated as required to synthesize oligomers of varying length and complexity.

Such methods each concern chemical synthesis of chemical material, particularly synthesis incident to DNA probe analysis. There exists a significant need for rapid automated methods which can accomplish multiple or arrayed analysis of samples containing cellular material, i.e. material containing intact whole cells or recognizable cellular components such as would be present after lysing procedures and the like. Heretofore no method has been proposed which permits the rapid and/or automated analysis of such samples.

Automated arrayed analysis of samples containing cellular material would be highly advantageous in numerous situations particularly those involving the screening or evaluation of components for potential pharmaceutical activity or efficacy. Such materials include classic pharmaceutical materials such as antibiotics. Heretofore the dispensing of potential components into contact with samples containing cellular material has been accomplished essentially by hand using various pipetting techniques. Dispensing of potential pharmaceutically active components as well as markers, indicators or other suitable analytical reagents to ascertain efficacy and/or activity into samples containing cellular material has generally been a time-consuming manual task. Such procedures have the risk of high rates of dispensing error. Additionally, the manual time involved can severely limit the number of variations and iterations employed. Thus crucial data can be omitted or overlooked due to gaps in analysis as well as errors in preparation.

It should also be noted that the amounts of reagent and cellular material employed in each iterative analysis can be quite large due to the limits to accuracy of manual dispensing techniques. In many circumstances relatively large sample volumes must be employed and associated volumes of test materials such as costly pharmaceutical materials must be obtained or synthesized in order to determine efficacy or activity. Thus, it would be highly desirable to provide a method and device which would permit reduction in volumes necessary to accomplish effective automated evaluation and screening of potential pharmaceutically active materials.

While ink jet technology has been proposed in chemical synthetic processes such as oligonucleotide synthesis, it has not been proposed or employed for use in conjunction with sample materials containing intact cellular material or identifiable components derived from cellular materials. Without being bound to any theory, it is believed that the sensitive and potentially fragile nature of compounds such as cellular material and potential pharmaceutically active materials mitigated against effective administration of potential agents to a suitable sample or plurality of sample by means of a droplet generator such as an ink jet device.

It should also be noted that evaluation of potential pharmaceutically active materials can require the use of precisely synthesized materials. Such materials are manufactured, stored and used under exacting standards of purity, cleanliness and the like. It would be highly advantageous to ship, store and dispense potential pharmaceutically active materials in suitable single use containers to ensure quality and purity. It would also be desirable that this container possess capability for on-demand dispensing of potentially multiple materials contained inside. Finally it would be advantageous to provide cartridges having means for capturing and storing data relevant to the container contents and relevant to dispensing protocols. It is also desirable that this data be interactively available to control means on the test apparatus and/or any diagnostic or analytic devices associated therewith.

Thus there exists a need for a method for analyzing substances containing cellular material; particularly for ascertaining the potential pharmaceutical activity of test components vis a vis such samples. There also exists a need for a test apparatus capable of ascertaining the effects of at least one potential pharmaceutically active agent on at least one substance containing cellular material as well as a need for a method for performing automated arrayed analysis on biologically derived samples containing cellular material. There also exists a need for single use cartridges containing at least one potential pharmaceutically active agent for use in such devices and methods.

### SUMMARY OF THE INVENTION

The present invention is directed to an automated method for analyzing substances containing cellular material, particularly pertaining to the screening of potentially pharmaceutically active components against biological samples as well as to a device for implementing such procedures. The method of the present invention includes a step in which at least one liquid jetting device is activated to dispense a first defined volume containing at least one potential pharmaceutically active agent into contact with a defined volume of a substance containing cellular material. Data pertaining to changes detected in the defined volume of the substance containing cellular material triggered by introduction of the first defined volume are captured for suitable review and interpretation.

The method of the present invention may be implemented by a suitable test apparatus which includes at least one automated liquid ejecting device. The potential pharmaceutically active agent may be contained in and delivered from a cartridge removably received relative to the test apparatus.

Other applications of the present invention will become apparent to those skilled in the art when the following description of the best mode contemplated for practicing the invention is read in conjunction with the accompanying drawings.

### BRIEF DESCRIPTION OF THE DRAWINGS

The description herein makes reference to the accompanying drawings wherein like reference numerals refer to like parts throughout the several views, and wherein:

FIG. 1 is a process diagram of the method of the present invention;

FIG. 2 is a block diagram of the test system of the present invention;

FIG. 3 is a diagram of the preferred cartridge system of the present invention;

FIGS. 4 and 5 are generalized drawings of droplet generating devices useful in the method and test apparatus of the present invention;

FIG. 4A is a schematic representation of a drop on demand generating apparatus as can be employed in the process and test device of the present invention, illustrating a removable cartridge and printhead each of which contains an optional memory storage device; and

FIG. 4B depicts a greatly enlarged view of an electrical memory storage device; and

### DESCRIPTION OF THE PREFERRED EMBODIMENT

The present invention provides a method for analyzing substances containing cellular material as well as a device for accomplishing the same. Even more particularly the present invention provides a method and device whereby various potential materials and compounds having unknown potential pharmaceutical or bio-active effects can be evaluated against specimens containing cellular material of interest.

The process of the present invention is preferably performed on a plurality of specimens each of which contain at least one target cell of interest in order to ascertain the effect of at least one potential pharmaceutically active agent on the cellular material in question. The term “potential pharmaceutically active agent” as used herein is defined as materials having known or suspected pharmacological effect as well as heretofore untested materials of interest. While pharmaceutics typically relates to the preparation and dispensing of drugs, the potential pharmaceutically active agent may be more broadly evaluated for bioactivity, i.e. the effect that a substance or agent has on or in living tissues or individual cells. Thus, it is within the purview of this invention that the material be potentially biocidal; i.e. capable of killing cells on a surface or the like. It is also within the purview of this invention that the potential pharmaceutically active agent be at least one cellularly active protein having potential therapeutic activity.

As broadly construed, the present invention is directed to an automated method for analyzing substances containing cellular material in which at least one liquid jetting device is activated to dispense a first defined volume containing at least one potential pharmaceutically active agent into contact with a defined volume of the substance containing cellular material. Data pertaining to changes detected in the defined volume of the substance containing cellular material triggered by introduction of the defined volume are captured. The captured data may be employed for independent interpretation subsequent to the process of the present invention by any suitable method. It is also within the purview of this invention that the captured data be interactively employed to recalibrate the liquid jetting device so as to dispense a second volume (or array of volumes) containing at least one potential pharmaceutically active agent into contact with defined volumes of the substance containing cellular material. The potential pharmaceutically active agent may be the same as that applied in the first volume or may be different in type, concentration, etc., depending upon the analysis being conducted.

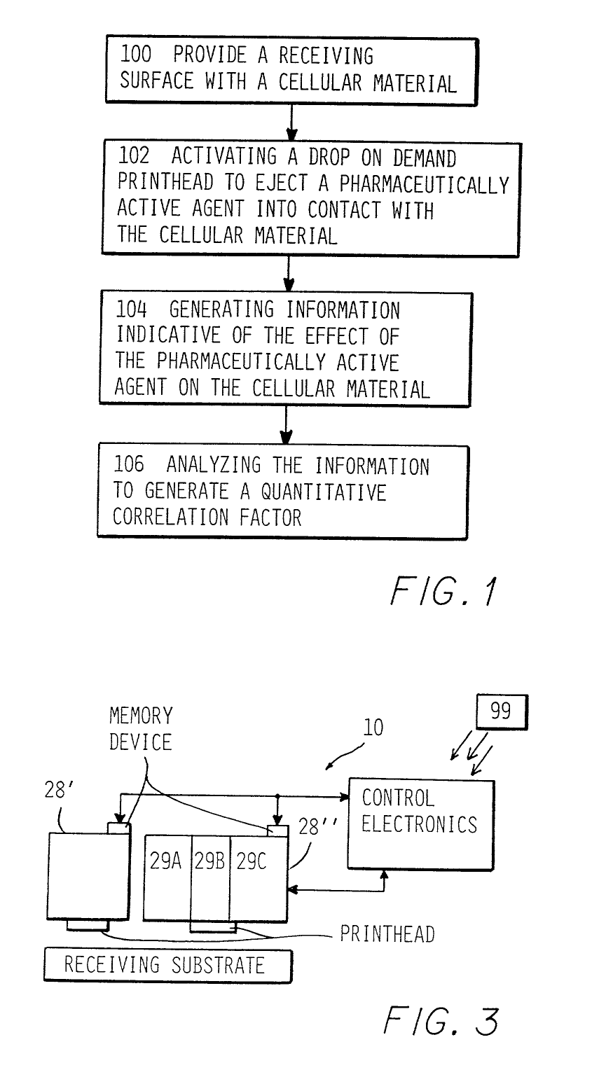

As broadly construed, the method of the present invention is set forth in FIG. 1. As indicated by 100, a receiving surface is provided with the cellular material. In one embodiment, the receiving surface arrives from a supplier with the material already in place. In a second embodiment, the operator of system 10 dispenses the cellular material onto the receiving surface. In a third embodiment, the cellular material is dispensed in two dimensional rectangular array.

As indicated by 102, the printhead(s) dispense pharmaceutical active agent(s) onto the cellular material. In the case of the third embodiment mentioned above, the amount and/or type of pharmaceutical agent varies across the array to enable an experimental design such as a factorial experiment that allows the sensitivity to the amount and/or type of the agent to be determined.

Prior to step 104, there can be a delay that varies from zero time to days or more to allow culturing of the cellular material and/or effects of the pharmaceutical active agent to take place.

As indicated by 104, information indicative of the effect of the pharmaceutically active agent is generated. This can be done manually by looking under a microscope and/or with an automated test system such as a vision system. The resultant information can take on a number of forms, such as a series of photographs, a data set, and/or qualitative observations.

As indicated by 106, the information from step 104 is analyzed. The method of analysis could take on a number of forms. In the case of the series of photographs or visual observations, the analysis could be anecdotal. For example, this could amount to “this combination of agents works best.” In the case of data gathering, the result might be more quantitative, such as a factorial analysis that would tend to generate a series of equations quantifying the results.

The flow chart above indicates how the system is utilized. It is configured by installing or calling up (such as by a menu) a particular control program, providing a particular cellular sample(s), and then installing particular cartridges containing the pharmaceutically active agent. The control electronics can be utilized to make sure that a particular experimental objective to be carried out will be met via the installed components.

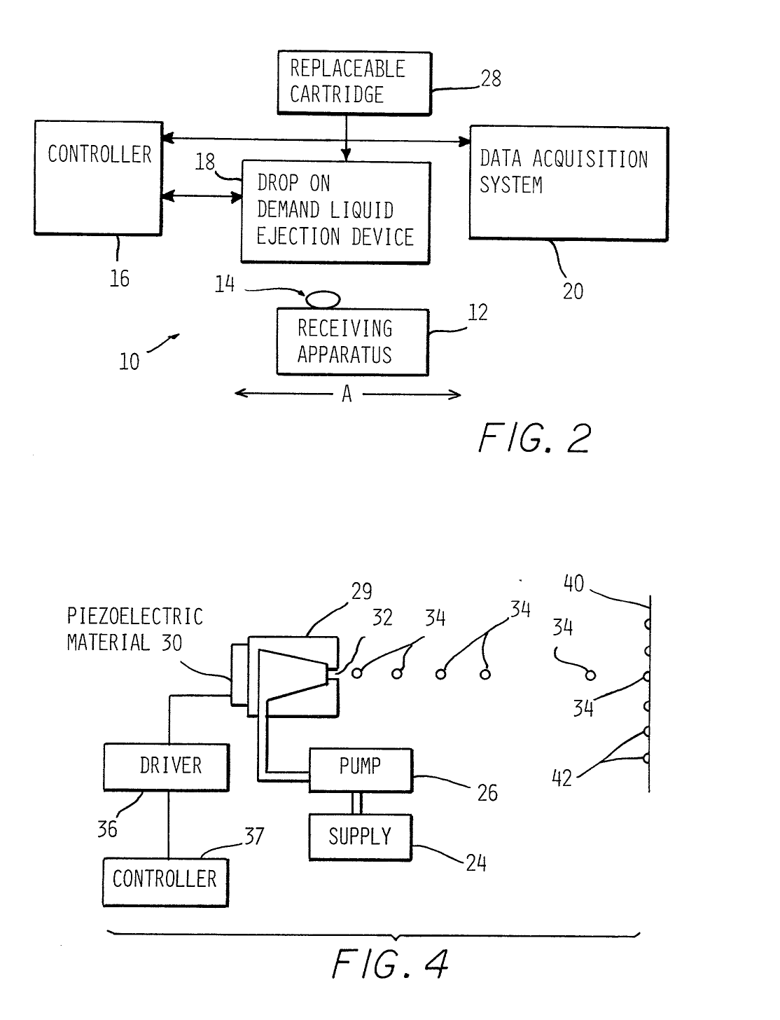

Referring to FIG. 2, a block diagram of the test system of the present invention is depicted as reference numeral 10. A receiving apparatus 12 typically holds the biological sample 14 to be studied. The biological sample 14 which is suitable for study in the method of the present invention is a substance or substances containing cellular material. The biological sample 14 to be studied may be present on the receiving apparatus as a single droplet or may be present as a series or array of discrete droplets or units. These units may be identical in size, concentration and/or preparation or may vary in parameters such as volume, concentration, carrier media, and/or pretesting preparation as desired or required by a predetermined testing protocol. It is also within the purview of the present invention that the discrete units of the individual specimen be identical to maximize information and data on the potential pharmaceutically active material or materials under study.

Typically substance or substances containing cellular material are ones which contain particular cells of interest for which evaluation of potential pharmaceutically active material is sought. These cells of interest are typically referred to as target cells. The target cells of interest may be cells which typically occur in a complex biological systems such as a human body or the like. The target cells may be atypically occurring cells such as cancer cells as well as typically occurring cells such as blood cells. The cells of interest may be parasites or infectious entities such as bacteria, fungi or viruses present in a complex biological system. It is also contemplated that target cells of interest may be derived from other non-human life forms as well a simpler independent noncellular life forms such as bacteria, protozoa and the like. The target cells may be in an active or dormant state depending on the analysis to be conducted.

The target cells in the biological sample 14 may be present in a carrier media. The carrier media is generally a solid or liquid material in which the target cells are contained. Typically the carrier is an aqueous or organic liquid which is chosen for its ability to support the target cells and provide minimum interference to the analysis being performed. The substance in which the target cells are contained may be a biologically derived fluid such as plasma, urine, cerebral spinal fluid, saliva and the like either in its native state or processed to fix or/and or concentrate the target cells. Alternately the target cells of interest may be removed from their environment and placed in a suitable growth or support media to promote or maintain cellular activity as necessary and/or desirable.

In the process and device of the present invention, it is contemplated that the testing for pharmaceutical activity can be performed on biologically intact cells. The process can be performed on recognizable material from intact cells, for example mitochondria, golgi bodies, nuclei, nucleoli and the like. Such materials may be made available from previously intact cells for analysis and testing by any suitable method such as lysing and the like. It is contemplated that material derived from intact cells suitable for testing and analysis by the method and device of the present are those generally discernable by high resolution microscopy, including but not limited to, microscopic analysis such as scanning electron microscopy. The materials suitable for analysis by the method and device of the present invention are those having measurable masses greater than molecular levels.

The receiving apparatus 12 has a specimen receiving surface which is preferably chemically and biologically inert relative to the cellular material under study. If desired, the receiving surface may be coupled with suitable means for monitoring the weight and/or volume of the specimen contained thereon. Suitable monitoring means include electronic balance mechanisms capable of weighing and recording the weight of a given sample as well as volumetric measuring devices as described in U.S. Pat. No. 5,601,980 to Gordon et al, the specification of which is incorporated by reference herein. The receiving apparatus 12 is typically configured to hold multiple sample volumes in a one-dimensional or two dimensional array of discrete volumes of the substances containing cellular material involved in the desired analysis.

As shown in FIG. 2, the device 10 of the present invention also includes a controller 16 coupled to a liquid ejection device 18 such as a drop on demand liquid ejection device. The drop on demand liquid ejection device may be in suitable fluid communication with a cartridge 28 or other suitable receptacle containing at least one potential pharmaceutically active agent preferably in liquid form. Typically, it is preferred that the solution containing at least one potential pharmaceutically active agent be contained in a suitable cartridge which is removably positionable in the device 10. The removable cartridge 28 may have suitable memory associated therewith and will work interactively in the liquid ejection device 18 in a manner such as will be described in detail subsequently. In the preferred embodiment, the liquid ejection device 18 operates in cooperation with an electronically actuated printhead which is preferably integrally associated with cartridge 28. Suitable printheads are commonly used to eject ink in ink jet printing devices and include piezo and thermal ink jet printheads as well as continuous inkjet printheads. Integral cartridges are preferred in situations requiring simplicity, ease of use and maintenance of sterility or integrity of the material housed in the cartridge during shipping storage and use.

In the device of the present invention, printheads are employed to eject a liquid or liquids containing at least one potential pharmaceutically active and/or at least one bio-active agent or agents. “Pharmaceutically active agents”, as the term is employed herein, are individual chemical compounds, formulations or complexes for which pharmacological activity is to be ascertained. Such materials may exert either a negative or positive impact in cellular activity of the target cell or cell line. This is particularly true of effects on cells or cell lines found within a more complex biological system such as a multi-cellular organism. Examples of such impact include, but are not limited to, increases or decreases in cell division, increases or decreases in production of specific enzymes, changes in motility or the like. It is also within the purview of the present invention that more than one cellular activity change may come under study. Such knowledge can be advantageous in further development of the material in question as well as ascertaining utility of a compound in new or untried areas. “Bio-active agents” as used herein are broadly defined as those exhibiting positive or negative effect on more primitive single or multicellular organisms such as bacteria, fungi and the like typically considered to be contaminants on various surfaces or structures.

The liquid containing the at least one potential pharmaceutically active agent to be ejected may be any material for which evaluation is sought. Examples of classes of such liquids and materials include, but are not limited to, antibiotics, antiseptics, cancer therapeutic agents such as folate antagonists, anthracyclines, angiostasis agents and the like. Other examples of potential pharmaceutically active agents include enzyme and hormone interactive materials, cholesterol lowering agents blood thinning agents and the like. It is contemplated that any liquid or solution deliverable by drop on demand ejection device 18 may be employed to evaluate bio-active and/or pharmaceutical effect.

As depicted in FIG. 2, in the preferred embodiment the controller 16 may be coupled to a suitable data acquisition system 20. The data acquisition system 20 suitable for use in the present system is one which will detect any changes characteristic to the cells and material under study. One such suitable data acquisition system 20 includes a suitable vision system configured to capture optical data such as visual data related to effects of the liquid or solution delivered from the drop on demand ejection device. Such changes in cells or cellular activity may include, for example, gross changes in color or opacity or minute, specific changes indicative of the health of individual cells.

The drop on demand liquid ejection device 18 is one which is capable of consistently emitting drops of a defined or known volume in a positionally accurate manner which follow sufficiently parallel drop trajectories so as to provide minimal spray, spattering of the like.

Referring now to FIG. 3, there is depicted a schematic rendition of what is considered to be the best mode of the intention. Preferably, the cartridge 28 has at least one reservoir containing a pharmaceutically active agent in which the reservoir containing pharmaceutically active agent is integral with a disposable printhead, preferably a drop on demand printhead. Such configuration is preferred as it eliminates issues associated with changing pharmaceuticals administered through the same printhead. In the preferred embodiment, capillary forces are used to refill the drop generators in each printhead. Such refill methods are known in the inkjet are and are present in replaceable cartridges commercially available from Hewlett Packard such as those designated HPC1823D or HP51645a.

It is within the purview of the present invention to employ more than one replaceable cartridge in performing the analytical procedures desired. In the embodiment depicted in FIG. 3, cartridge 28 contains a diluent or interlayer material and cartridge 28 contains three different potential pharmaceutically active agents in chambers 29a, 29b and 29c.

Each cartridge has an integral memory device that provides information the control electronics that is indicative of the cartridge contents. Each time a certain type or class of experiments is to be run, a new set of cartridges are loaded. The one-chamber and three-chamber cartridges above are only exemplary and it can be seen that an immense number different evaluations can be performed with the same system 10, depending upon the cartridges installed.

The controller 16 into which is installed a media such as a CD-rom that contains information pertaining to a particular experiment. The electronics verify via the memory devices whether the installed cartridges correspond to the experiment being performed. It not, a warning can be displayed to the user of system 10 of an improper match of cartridges and the experiment. Thus, system 10 is a flexible system that can be configured for a particular type of test via media and the cartridges.

A piezoelectric drop on demand chemical jetting device is depicted in FIG. 4. It will be appreciated that chemical jetting apparatus suitable for use in the present invention has essentially similarities to apparatus used in “inkjet printing.” Various inkjet printers are known per se. Details relating to inkjet technology, methodologies for employment of inkjets and to apparatus for use are known to the skilled artisan.

Alternate chemical jetting configurations suitable for use in the present invention would, preferably, include a plurality of nozzles with suitable associated firing chamber capable of repetitive controlled firing. Referring to FIG. 4, a chemical jetting device having a piezoelectric actuation system is depicted. A source supply 24 of potential pharmaceutically active agent is provided through a pumping means 26 to a chamber 29 in mechanical communication with a piezoelectric material 30. The chamber 29 is provided with one or more orifices 32 through which droplets 34 of the potential pharmaceutically active agent may be ejected through the controlled pumping action of the piezoelectric material 30. The piezoelectric device is controlled by a driver 36 which is controlled by a suitable on board controller 37.

The individual droplets 34 can be directed to selected locations on the surface 40 of the receiving apparatus 12. It will be appreciated in the device depicted in FIG. 4, that droplets are provided only when actually required for positioning on the surface 40 of the receiving apparatus 12 occupied by at least one volume 42 of the solution containing cellular material. In the preferred embodiment, the drop on demand device may include means for physically moving the source of droplets, such as the droplet generator, with respect to surface 40 of the receiving apparatus. It is also within the purview of this invention to maintain a fixed droplet generator together with a movable test surface such as denoted in the direction denoted in “A” in FIG. 2. In either manner, the droplets of potential pharmaceutically active agent are delivered to precise locations on the surface in an image wise, preselected fashion. It is to be understood that other firing and targeting mechanisms may be employed which would be compatible with the potential pharmaceutically active material to be delivered.

Without being bound to any theory it is believed that efficient and intimate contact and mixture of each volume 42 of substance containing cellular material and the associated droplet 34 containing at least one potential pharmaceutically active agent occurs as a result of the impetus provided by the piezoelectric material 30 or other suitable firing device. The firing device of choice is one which can force the material through the orifice with sufficient kinetic energy to contact and interact with the defined discrete volumes of solution containing cellular material contained on the test surface.

The controller 16 of test apparatus 10 is one capable of providing controlled positioning of the liquid ejection device 18 with respect to the receiving apparatus 12. Among other functions, the controller 16 of the apparatus 10 can control the vertical Z-distance of the drop ejector from each individual sample containing cellular material. Preferably, it would also impart relative motion to the drop on demand ejection device 18 with respect to the receiving apparatus 12 to allow the ejection chamber to access regions of the receiving apparatus 14 which are to receive selective deposition of the material containing the potential pharmaceutically active agent.

In this embodiment, the supply source 24 of the potential pharmaceutically active agent is maintained in a replaceable cartridge 28 having an appropriate supply vessel such as container 44. The may be a separate element which is capable of removable fluid contact with the replaceable cartridge 28. In such instances, the printhead may be an essentially permanent element associated wit the test device or may be separately removable as depicted in FIGS. 3A and 3B.

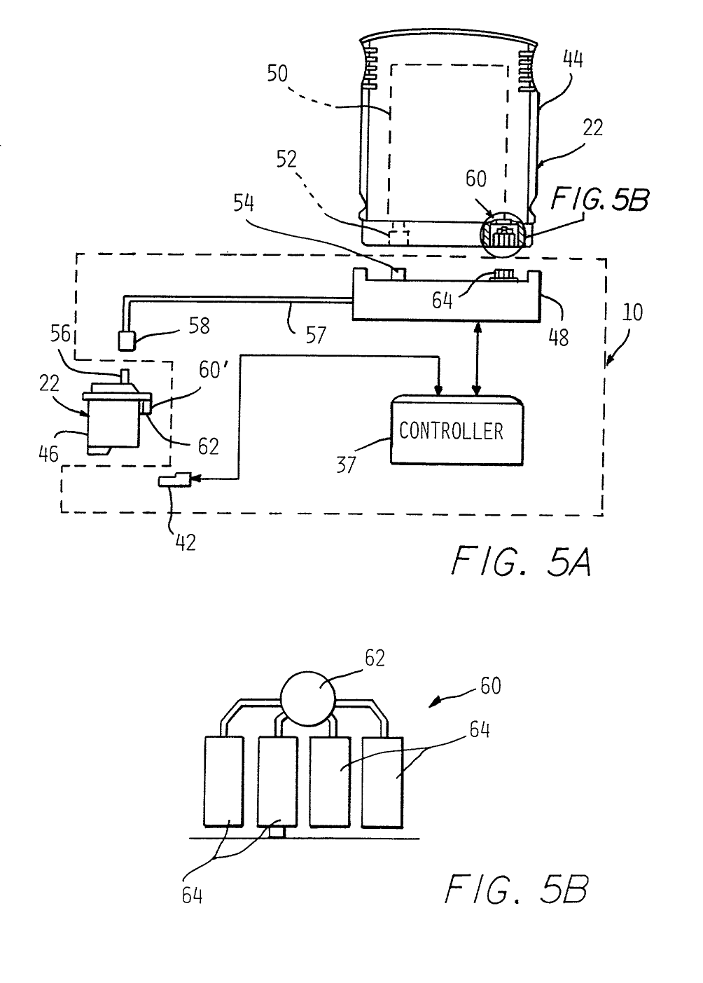

FIGS. 5A and 5B depict a simplified schematic representation of the automated testing system 10 of the present invention as shown in FIG. 2 having separately replaceable printheads and cartridges. FIGS. 5A and 5B are simplified to illustrate a single container 44 and a single dispensing system for single potential pharmaceutically active agent. In situations where dispensing of more than one potential pharmaceutically active agent alone or in combination with regents indicators or the like is desired, the container 44 may have a plurality of suitable compartments.

The test apparatus 10 of the present invention includes an container receiving station 48 in association with a controller such as controller 37. When the container 28 is properly inserted into the container receiving station 48, an electrical and fluidic coupling is established between the container 44 and the printhead. The fluidic coupling allows the potential pharmaceutically active agent stored within the container 44 to be provided to suitable printhead 46. The electrical coupling allows information to be passed between the replaceable container 44 and the test apparatus portion 10 as needed to provide pertinent information regarding general operational information as well as potentially scientifically relevant data such as shelf life, lot number as well as detailed chemical and biological information.

In the preferred embodiment, it is envisioned that controller 37 controls the transfer of information between the test apparatus 10 and the container 44. Finally the controller 37 also interactively cooperates with master controller 16 to control relative movement of the container 44 and the test surface 40 as well as selectively activating the container 44 and connected printhead to dispense defined volumes of potential pharmaceutically active agent into contact with the individual units of samples containing cellular material.

The container 44 typically includes means for storing at least one potential pharmaceutically active agent therein. Storage chambers in container 44 typically provide capacity for maintaining a plurality of discrete materials in storage isolated from one another until dispensing is required. The container 44 may include suitable mixing means (not shown) upstream of fluid outlet 52. Alternatively mixing may occur as necessary or required at the printhead in suitable manner. As depicted in FIGS. 5A and 5B, the fluid outlet or outlets 52 are configured for connection to at least one complimentary fluid inlet 54 associated with the cartridge receiving station 48.

As shown in FIGS. 5A and 5B the printhead 46 includes a fluid inlet 56 configured for connection to a complimentary fluid outlet 58 associated in the dispensing portion. The printhead 46, when properly inserted into the test apparatus, establishes fluid communication between the printhead and the container by way of a flexible fluid conduit 57.

Each replaceable printing component such as printhead and container 44 may include at least one suitable information storage device such as electrical storage device or memory 60, 60 for storing information relating to the associated replaceable component. A plurality of electrical contacts or linking portions 64 may be provided each of which is electrically connected to the electrical storage device 62. When the container 44 is properly inserted in the cartridge receiving station 48 of the test apparatus 10, each of the plurality of electrical contacts engage a corresponding plurality of electrical contacts 64 associated with the receiving station 48. Each of the plurality of electrical contacts 64 associated with the cartridge receiving station 48 are electrically connected to the controller 37 by a plurality of electrical conductors. With proper insertion of the cartridge 28 into the cartridge receiving station 48, the memory associated with the cartridge is electrically connected to the controller 37 allowing information to be transferred between the container 44 and the test apparatus 10. Similar communication can be achieved between the printhead and controller. While the foregoing discussion has been directed to a system having a discrete moveable printhead separate from the container, it is to be understood that a cartridge in which the two elements are integrally joined is preferred.

In the preferred embodiment, it is anticipated that the cartridge 28 having integral printhead or container 44 independent of printhead would be both removable and consumable. Thus, the potential pharmaceutically active agent could be prepackaged under suitable and applicable pharmaceutical standards and shipped to an end user for testing of biological samples. Single use cartridges would enhance ease of use and help to insure consistency and reproducibility. Once the contents have been used, the cartridges may be returned for refill or disposed of in a manner consistent with recognized acceptable standards. The contents of a given cartridge may be a standard component cohort as would be employed in matters of routine or standardized analysis. It is also anticipated that cartridge contents may be customized to meet the requirements of more unique analytical procedures.

While contents have been referred to as at least one single pharmaceutically active agent, it is also considered to be within the purview of this invention that the cartridge and/or container be configured so as to contain a plurality of discrete agents to be dispensed. These agents can include, but are not limited to, additional potential pharmaceutically active agents as well as indicators, markers and the like. Preferably the various components of the cartridge are maintained in suitable discrete chambers located therein. The multicomponent cartridge has suitable means for conveying the desired component to the printhead for controlled dispatch in a sequence predetermined and implemented by the controller. Additionally, the optional memory storage device associated with the removable cartridge could contain detailed information regarding formulation, chemical analysis, lot number, expiration date and the like of each material contained therein. Such data can be of great importance in interpreting results which accrue to biological samples such as those containing cellular material. The cartridge memory device can also include information regarding details of the performance of the cartridge as well as information interface with the associated printhead. Examples of such information include, but are not limited to, material viscosity, precise ejected drop volume, ejection velocity, and the like. It can be appreciated that such detail can provide powerful and meaningful information necessary for the interpretation of results which are ultimately gathered and recorded. Memory systems of this source are further detailed in U.S. Pat. No. 6,039,430 to Helterline, et al., the specification of which is incorporated by reference herein.

The present invention is also directed to a method for analyzing substances containing cellular material. As broadly construed, the method includes the steps of activating at least one liquid jetting device to dispense a first defined volume containing at least one potential pharmaceutically active agent into contact with a defined volume of a biological substance containing cellular material. Data pertaining to changes detected in the defined volume of the substance containing cellular material which correlate to or are triggered by the introduction of the first defined volume is detected and captured in a suitable manner.

Typically, the substance containing cellular material is maintained in contact with a suitable testing substrate. The suitable testing substrate has a contact surface which is reactively inert to interaction with the cellular material under study. The term “reactively inert” as used herein is defined as a material which can maintain the defined volume in a discrete collected manner without exerting or initiating chemical or physical alteration of the substance containing cellular material. Typically, each defined volume of the substance containing cellular material is maintained on the surface of the testing substrate. However, the testing substrate contact surface may include features which enhance surface tension or provide other mechanisms for maintaining the defined volume in a discrete location on the contact surface.

While the automated method of the present invention can be performed on an individual defined volume of a substance containing cellular material, the automated method of the present invention is advantageously employed upon multiple discrete volumes of the substance containing cellular material. Thus, in the preferred embodiment, a plurality of defined volume are positioned on the contact surface of the testing substrate in a linear or arrayed manner. While the defined volume of the substance containing cellular material may have any suitable volume capable of rendering and providing effective test results, it is preferred that each defined volume be as small as possible to minimize waste and to facilitate safe and effective disposal of any hazardous biological or chemical material once the test is complete.

The automated method of the present invention can be performed effectively on a plurality of defined volumes and/or concentrations of the substance containing cellular material. These defined volumes positioned on the contact surface of the test substrate may be essentially identical to one another. In such cases, characteristics of the potential pharmaceutically active agent to be administered could be varied to provide a continuum of potential results. It is also possible that the content or specific characteristics of the substance containing cellular material may vary from individual volume to individual volume in a known predetermined manner. This variation could include, but is not limited to characteristics such as cellular concentration, cellular age, concentration or contents of the carrier material, concentration characteristics of any indicator material present in the carrier material, or the like.

In the automated method of the present invention, at least one jetting device dispenses at least one potential pharmaceutically active agent into contact with the individual volume(s) of the substance containing cellular material. In such automated methods, the preferred jetting device will be one which provides little or no interferences with the activity of potential performance of the pharmaceutical agent. Thus, in situations where the potential pharmaceutically active agent is thermally stable, jetting devices such as those commonly known in thermal inkjet printing art may be employed. The method of the present invention may also utilize a piezoelectrically driven jetting device to administer the potential pharmaceutically active agent. It is generally preferred that either device be configured to provide drop on demand liquid jetting capabilities and be capable of selectively and precisely administering defined volumes of the potential pharmaceutically active agent into contact with each defined volume of the substance containing cellular material which is present on the contact surface of the testing substrate. The method of delivery and the parameters regarding volume and administration are generally interactively controlled between a master controller present on the testing device and any optional on-board memory and/or subcontrollers present in the jetting device itself. Typically, at least one potential pharmaceutically active agent may be dispensed in quantities which vary compositionally and/or concentrationally across the individual volumes of the substance containing cellular material.

It is within the purview of this invention to include means for administering quantities of additional potential pharmaceutically active agent into contact with the defined volumes of the substance to be tested. The additional quantities of potential pharmaceutically active agents can be a different cohort of potential pharmaceutically active agents or can be similar or identical to pharmaceutically active agents previously dispensed as would be dictated by parameters of the investigation being conducted. Additionally, materials which would be adjuncts, catalysts, accelerators or the like, could be administered in tandem with the potential pharmaceutically active agent or agents in various combinations and sequences determine the net effect of such combinations on the substance in question. Finally, various indicators or optical enhancers can be administered through the jetting device to mark or elucidate the cellular response to the pharmaceutically active agent or agents administered. Such materials are preferably contained in at least one cartridge capable of removable insertion into fluid contact with the associated test apparatus. Thus, it can be readily appreciated given the foregoing that multi-modal strategies can be quickly and readily evaluated for potential cellular response. Given the precision and automation with which the materials are administered, it is also possible to more precisely evaluate response time, and to control or integrate details regarding surrounding environmental characteristics and the like.

The biological substances containing cellular material under study can be positioned on the testing substrate by any suitable method. These include manual positioning as well as positioning by suitable liquid jetting devices. Each defined volume of the substance containing cellular material can be present in any suitable array which is capable of yielding statistically viable data. The individual samples can be arranged in a defined two-dimensional array as well as in an iterative linear array as required by the particular investigational study.

Typically, in performing the method of the present invention, the operator uses a test apparatus such as the test apparatus 10 defined previously. Pertinent data concerning aspects of the experiment to be performed is entered into the test apparatus 10 through any suitable data entry means. At least one cartridge containing the potential pharmaceutically active agent under investigation is installed in the test apparatus 10 in a manner which establishes fluid and, optionally, electronic communication between the test apparatus and the cartridge.

Defined volumes of the substance containing cellular material are positioned on the test surface. The positioning may be in any manner which will permit linear or dimensional array of the defined volumes. The biological material is dispensed by any suitable means.

In the method of the present invention, the potential pharmaceutically active agent is dispensed into contact with each defined volume of the biological substance containing cellular material. In situations where the defined volumes of cellular material are present in an array, the quantity, concentration, ordering and/or types of pharmaceutical materials and adjunct can be varied to yield arrayed results as required by the particular investigation.

The samples are analyzed to determine detectable changes in some or all of the samples tested. Preferably, analysis occurs by analytical systems which are compatible with the automated nature of the testing method of the present invention. Such systems includes data acquisition or vision system. Such vision system can include high resolution microscope units coupled to automated pattern and/or color recognition systems as would be known in the art. Such vision systems can monitor indicators of cellular health. Such systems can collect resultant data in an automated and efficient manner which would allow the rapid and automated testing of large numbers of different cells and pharmaceutical mixtures. The vision system of choice can back light the substrate in situations where the test substrate is clear. In situations where the test substrate is reflective, the vision system can utilize overhead lighting to accomplish analysis.

It is to be understood that the data acquisition system may employ other analytical systems unrelated to vision acquisition systems. Such systems may include laser emitters and detective pairs, infrared analysis, x-ray fluorescence and the like.

Data acquired by one or more analytical methods can be analyzed by any suitable method to ascertain effectiveness and the like. The data acquired can be analyzed in a static method subsequent to test completion. It is also contemplated that acquired data can be employed in an interactive or dynamic fashion to alter dispensing patterns in future test specimens in an interactive manner as would occur in an ongoing factorial analysis protocol.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. An automated method for analyzing substances containing cellular material, the method comprising the steps of:
activating a test apparatus having at least one liquid jetting device to dispense a first defined volume containing at least one potential pharmaceutically active agent into contact with a defined volume of a substance containing cellular material; and
capturing data pertaining to changes detected in the defined volume of the substance containing cellular material triggered by introduction of the first defined volume.

2. The automated method of claim 1 wherein the jetting device comprises at least one cartridge containing at least one potential pharmaceutically active agent removably associatable with the jetting device.

3. The automated method of claim 1 wherein the defined volume of the substance containing cellular material is maintained in contact with a suitable testing substrate, the suitable testing substrate having a contact surface which is reactively inert to interaction with the cellular material under study.

4. The automated method of claim 3 wherein the defined volume of the substance containing cellular material comprises a plurality of individual volumes, wherein each individual volume is between about 1 and about 500 picoliters and wherein characteristics of the substance containing cellular material may vary from individual volume to individual volume in a known predetermined manner.

5. The automated method of claim 4 wherein the at least one jetting device dispenses varying quantities of at least one potential pharmaceutically active agent into contact with the individual volume of the substance containing cellular material.

6. The automated method of claim 4 wherein the at least one jetting device dispenses a quantity of at least one potential pharmaceutically active agent into contact with selected individual volumes present, the dispensed quantity varying compositionally across the individual volumes of the substance containing cellular material.

7. The automated method of claim 1 wherein the defined volume of a substance containing cellular material is present as a plurality of individual samples arranged in an array capable of yielding statistically viable data.

8. The automated method of claim 7 wherein the individual samples are arranged in a defined two-dimensional array.

9. The automated method of claim 7 wherein the individual samples are arranged in an iterative linear array.

10. The automated method of claim 1 further comprising the step of interactively activating at least one second liquid jetting device to dispense a second defined volume of a potential chemically active substance into contact with the defined volume of the substance containing cellular material.

11. A test apparatus for ascertaining effects of at least one potential pharmaceutically active agent on a substance containing cellular material, the test apparatus comprising:
a test surface configured to receive at least one discrete volume of the substance containing cellular material; and
at least one automated drop on demand liquid ejection device capable of administering at least one potential pharmaceutically active agent into contact with the discrete volume of the substance containing cellular material present on the receiving means.

12. The test device of claim 11 wherein the test surface has at least one face adapted to receive the substance, the receiving face composed of a material which is essentially non-reactive with the substance positioned thereon.

13. The test device of claim 12 wherein the planar surface has defined therein a plurality of receiving positions configured in an arrayed pattern.

14. The test device of claim 11 wherein further comprising a controller coupled to the drop on demand liquid ejection device to provide controlled deposition of the potential pharmaceutically active agent into contact with the substance containing cellular material.

15. The test device of claim 14 wherein the drop on demand liquid ejection device is an electronically actuated printhead.

16. The test device of claim 15 wherein the electronically actuated printhead includes at least one multichamber printhead, the multichamber printhead in fluid communication with a reservoir containing at least one potential pharmaceutically active agent.

17. The test device of claim 16 wherein the reservoir containing at least one potential pharmaceutically active agent is a cartridge removably positionable in fluid contact in the test device, the cartridge maintaining the at least one potential pharmaceutically active agent in discrete and isolated position therein until positioned relative to the test device.

18. The test device of claim 14 further comprising:
at least one detection device, the detection device capable of ascertaining detectable changes in the discrete volume of the substance containing cellular material subsequent to introduction of the potential pharmaceutically active agent; and
at least one data capture device, the data capture device capable of recording data pertaining to the material dispensed from the drop on demand liquid ejection device, wherein the data capture device is in communication with the multichamber printhead.

19. A replaceable cartridge component for use in a test apparatus for ascertaining effects of at least one potential pharmaceutically active agent on a biological substance, the test apparatus including at least one drop on demand liquid ejection device the replaceable cartridge comprising:
a container having an interior volume containing at least one pharmaceutically active agent; and
a printhead in fluid communication with the container, the printhead capable of dispensing the at least one pharmaceutically active agent in the container.

20. The replaceable cartridge component of claim 19 further comprising at least one memory storage device capable of capturing and maintaining information pertaining to cartridge function and contents and communicating said captured information to the test apparatus.

21. The replaceable cartridge component of claim 20 further comprising control electronics, the control electronics capable of converting received information into control output pertinent to at least one aspect of the effect analysis.

22. A replaceable cartridge component for use in a test apparatus for ascertaining effects of at least one potential pharmaceutically active agent on a biological substance, the test apparatus including at least one drop on demand liquid ejection device the replaceable cartridge comprising:
a container having an interior volume containing at least one pharmaceutically active agent; and
a memory storage device, the memory storage capable of storing information pertinent to at least one aspect of test apparatus function.

23. The replaceable cartridge component of claim 22 wherein the information stored in the memory storage device comprises data pertaining to cartridge function and contents.

24. The replaceable cartridge of claim 22 further comprising a printhead in fluid communication with the container, the printhead capable of dispensing the at least one pharmaceutically active agent in the container.

25. A method for performing arrayed analysis on a biologically derived material containing cellular material, the method comprising the steps of:
activating at least one drop on demand liquid ejection device to dispense a defined volume containing at least one potential pharmaceutically active agent into contact with a plurality of defined volumes of a substance containing cellular material;
capturing data pertaining to changes detected in the defined volume of the substance containing cellular material triggered by introduction of the first defined volume into each defined volume containing cellular material for suitable review and interpretation; and
upon interpretation of the captured data, altering dispensation of the potential pharmaceutically active material in an iterative manner.

26. The method for performing arrayed analysis of claim 25 wherein the iterative variation is a function of an ongoing factorial analysis protocol.
